(19) United States
(12) Patent Application Publication
(10) Pub. No.: US 2024/0398617 A1
(43) Pub. Date: Dec. 5, 2024

(54) SYSTEMS FOR INTRAOCULAR SURGERY

(71) Applicant: Alcon Inc., Fribourg (CH)

(72) Inventors: Patrick TERRY, Goleta, CA (US); Sumit PALIWAL, Irvine, CA (US); Mark Andrew ZIELKE, Lake Forest, CA (US); Lance NOLLER, Yorba Linda, CA (US)

(21) Appl. No.: 18/677,567

(22) Filed: May 29, 2024

Related U.S. Application Data
(60) Provisional application No. 63/505,054, filed on May 30, 2023.

(51) Int. Cl. A61F 9/007; A61B 34/30; A61B 90/00

(52) U.S. Cl. CPC A61F 9/007; A61B 34/30; A61B 90/37; A61B 2217/005; A61B 2217/007

(57) ABSTRACT

A system for assisting a surgeon performing intraocular surgery on an eye of a medical patient includes a robotic arm and a tool assembly at a working end of the robotic arm. The tool assembly can include a proximal end mounted to the robotic arm's working end, a distal end, and a narrow elongate support member extending between the tool assembly's proximal and distal ends. A motion controller is operable to move the robotic arm to extend the distal end and at least a portion of the support member through an incision site in a surface of the patient's eye, thereby to locate a visualization element at the distal end of the tool member at a position inside the patient's eye.

### CROSS-REFERENCE TO RELATED APPLICATIONS

This application claims benefit of and priority to U.S. Provisional Patent Application No. 63/505,054, filed May 30, 2023, which is hereby assigned to the assignee hereof and hereby expressly incorporated by reference in its entirety as if fully set forth below and for all applicable purposes.

### INTRODUCTION

Glaucoma is a disease of the eye, frequently related to excess pressure within the eye's interior. High pressures within the eye can damage the optic nerve, which can lead to worsening vision and even blindness. Glaucoma is one of the most frequent causes of blindness in patients over the age of 60.

Mild glaucoma is sometimes treated through topical applications of appropriate medications. More severe glaucoma may frequently require surgery. These surgeries can include canaloplasty, trabeculectomy, and the placement of implants to increase the drainage of excess fluid from the eye's anterior chamber. These procedures can reduce fluid pressure fluid inside the eye, and hence the unwanted and damaging overpressure that might otherwise be applied to the optic nerve.

In some relatively new surgical techniques, called minimally invasive glaucoma surgeries (MIGS), an intraocular micro-stent may be placed at a target site on an interior surface inside the eye. A properly placed micro-stent enhances the travel of intraocular fluid, for example, out of the eye, through the trabecular meshwork, and into Schlemm's canal. Alternatively, certain MIGs procedures may rely on small incisions (and/or excisions) of intraocular tissues for facilitating improved aqueous outflow into the Schlemm's canal, or dilation of the Schlemm's canal via a viscoelastic.

The effectiveness of such surgeries depends, though, on the proper placement of intraocular incisions and/or the proper placement of the micro-stent drainage device. This frequently depends on the effectiveness with which the target site for placement of the intraocular incision or micro-stent can be visualized by the surgeon. This visualization can be particularly challenging in minimally invasive procedures, in which only a very small entry incision is made through the eye's outer surface for the insertion of tools and instruments. Further, the relevant structures of the eye are conventionally viewed via external instruments such as mirrors and prisms that provide only indirect visualization of the eye's structures and the target site at which the surgeon is attempting to make intraocular incisions and/or place the stent.

Accordingly, there is a need in the art for improved systems for performing minimally invasive intraocular surgeries, and in particular, systems for providing improved visualization of the eye's internal structures to facilitate the performance of such surgeries by surgeons.

### SUMMARY

Embodiments described in this disclosure relate generally to systems for assisting a surgeon performing ophthalmic (e.g., intraocular) surgery on an eye of a medical patient. Examples of such systems may include a robotic arm and a tool assembly at a working end of the robotic arm. The tool assembly can include a proximal end mounted to the robotic arm's working end, a distal end, and a narrow elongate support member extending between the tool assembly's proximal and distal ends. A motion controller may be operable to move the robotic arm to extend the distal end and at least a portion of the support member through an incision site in a surface of the patient's eye in order to position a visualization element at the distal end of the tool member at a desired position inside the patient's eye.

Certain embodiments described in this disclosure relate to robotic systems for integration with visualization and/or other surgical devices. Example surgical devices and robotics platforms are further described in U.S. Pat. No. 10,917,543 B2, which is herein incorporated by reference in its entirety.

In one embodiment, a system for intraocular surgery is provided, the system comprising: a robotic arm; a tool assembly at a working end of the robotic arm, the tool assembly comprising: a proximal end at the working end of the robotic arm; a distal end; a support member extending between the proximal end and the distal end of the tool assembly; and a visualization element at the distal end of the tool assembly; and a motion controller operable to move the robotic arm to extend the distal end and at least a portion of the support member into an eye of a medical patient through an incision site in a surface of the patient's eye, so that the visualization element is located at a position inside the patient's eye.

In another embodiment, a system for intraocular surgery is provided, the system comprising: an adjustable arm; a tool assembly coupled to a working end of the adjustable arm, the tool assembly comprising a visualization element at a distal end of the tool assembly; and a motion controller operable to move the adjustable arm to extend the distal end into an eye of a medical patient through an incision site in a surface of the patient's eye, so that the visualization element is located at a position inside the patient's eye, wherein: the adjustable arm is movable with at least six degrees of freedom, and wherein the motion controller is operable to move the adjustable arm so that motion of a portion of the support member is constrained at a virtual fixed point of rotation at the incision site in the patient's eye surface, and the motion controller is configured to receive input from a user of the system via a motion controller input device that includes at least one of a hand control, a foot control, or apparatus pre-programmed to effect a desired motion of the robotic arm.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a perspective view illustrating a surgery being performed in an operating environment with a surgical system, according to aspects of this disclosure.

FIG. 2 is a perspective view of a robotic arm for use with the surgical system of FIG. 1, according to certain aspects of this disclosure.

FIG. 3 depicts a tool assembly at a working end of the robotic arm of FIG. 2, according to certain aspects of the disclosure.

FIG. 4 is a schematic illustration of a tool assembly that is easily removable from a robotic arm of FIG. 2, according to certain aspects of the disclosure.

FIG. 5 is an enlarged perspective view of a distal end of a tool assembly like that shown in FIG. 3 or FIG. 4, according to certain aspects of the disclosure.

FIG. 6 is an end view of the distal end of the tool assembly of FIG. 5, illustrating four ports for working elements used in the tool assembly, according to certain aspects of the disclosure.

FIG. 7 is an end view of a distal end of another tool assembly, in which a ring-like illumination element surrounds three ports for working elements used in the tool assembly, according to certain aspects of the disclosure.

FIG. 8 is a block diagram schematically illustrating components of the surgical system, according to certain aspects of the disclosure.

FIG. 9 is a perspective view showing a distal end of a tool assembly inserted through an incision in a patient's eye during a surgery using the surgical system, according to certain aspects of the disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

This disclosure relates generally to systems for assisting ophthalmic surgeons in performing ophthalmic (e.g., intraocular) surgeries. These surgeries may include, for example but not by way of limitation, the placement of intraocular stents or other drainage devices for relieving intraocular pressure associated with glaucoma.

Various examples will now be described more fully with reference to the accompanying drawings. Systems like those disclosed here may, however, be embodied in various different forms and should not be construed as limited to the examples set forth here.

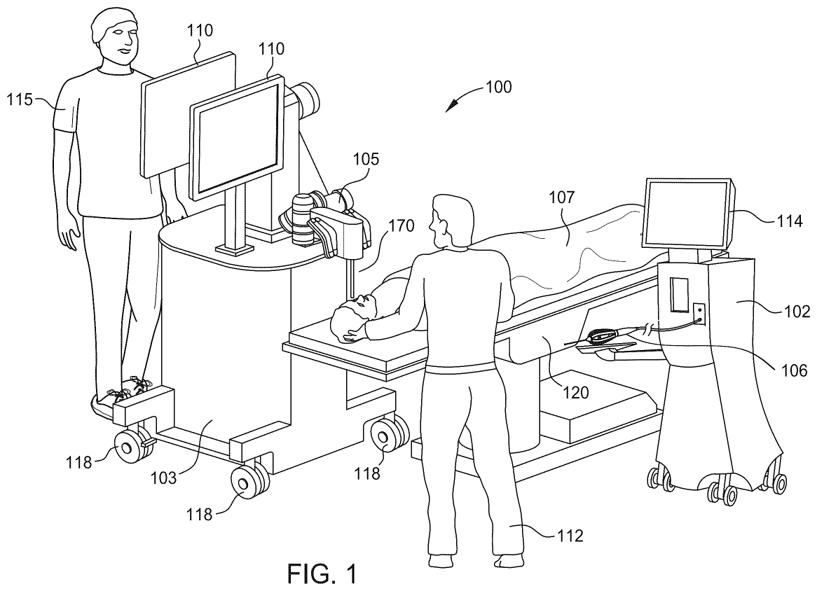

FIG. 1 depicts a system 100 for performing an intraocular surgery. The system 100 includes a surgical console 102 and a robotic arm 105. The surgical console 102 may include, or be operably coupled to (e.g., physically or wirelessly), one or more modules, systems, devices, and/or surgical tools for performing one or more surgical procedures, including MIGS procedures. For example, in certain embodiments, the surgical console 102 comprises one or more ports for coupling a surgical tool 106 to an internal fluid source, vacuum source, and/or driver. In certain embodiments, the surgical console 102 may be in physical or wireless communication with the robotic arm 105.

The robotic arm 105 is operable to hold, move, and insert a tool assembly into and within an interior volume of an eye of a medical patient 107. Although it is currently contemplated that the medical patient will most frequently be a human patient, in veterinary applications the “patient” can be a non-human animal, and the term as it appears in this disclosure should be read to encompass either possibility.

The robotic arm 105 may be supported by any suitable device or system within an operating environment. In the example of FIG. 1, the robotic arm 105 is supported by a cart 103. The cart 103 can itself be fixed or movable with respect to a structure for supporting the patient 107. FIG. 1 depicts an installation in which the cart 103 is supported on wheels 118 that allow the cart 103 to be rolled into position with respect to a patient support table 120, which is illustrated as fixed to the floor of a surgical suite in which the procedure is performed. In other embodiments, however, the robotic arm 105 may be supported by the surgical console 102 or another supporting structure within the operating environment. In particular embodiments, the robotic arm 105 might, for example, be supported on a movable cart (e.g., a device, case, or supply cart), the floor, ceiling, or a wall of a surgical suite or another room in which the equipment is located, a bed or a similar structure on which the patient is placed for treatment, or any other suitable location. In certain embodiments, the robotic arm 105 is movably attached to a track or rail upon which the robotic arm 105 may translate. Such track or rail may be disposed on, for example, the surgical console 102, a movable cart, the floor, ceiling, or a wall of a surgical suite or another room.

The system 100 may further include one or more video display monitors 110, or other visualization devices such as augmented reality (AR) or virtual reality (VR) headsets and the like, for delivering information and images to medical personnel in the course of a surgery. FIG. 1 illustrates a system with two display monitors 110 disposed on the cart 103 for use, for example, by a surgeon 112 and an assistant 115. The display monitors 110 can be communicatively coupled with the surgical console 102 (which may have its own display monitor 114), a visualization system within the operating environment, and/or the tool assembly supported by the robotic arm 105. In some embodiments, the display monitors 110 can receive information (e.g. surgical parameters) and/or images from the surgical console 102 and tool assembly, respectively, and display the information and images on the display monitors 110. The surgical console 102 can also send signals to the display monitors 110 for performing operations (e.g. starting and stopping video recording).

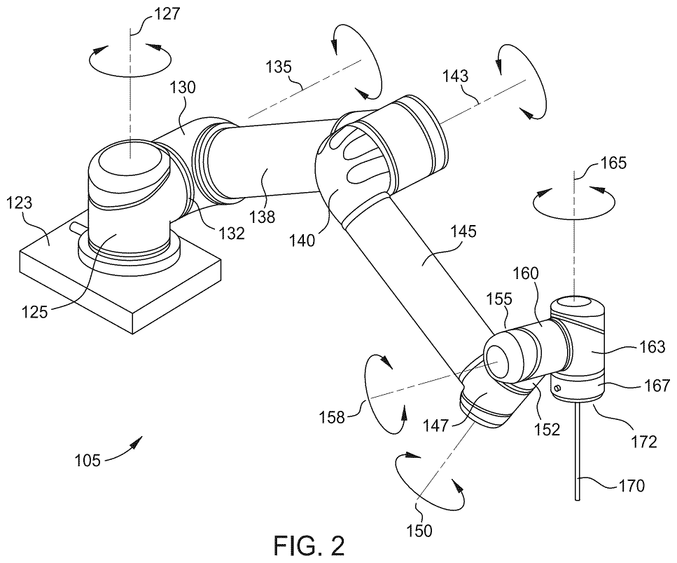

Turning now to FIG. 2, elements of the robotic arm 105 are illustrated in more detail. As shown, the robotic arm 105 includes a support base 123 which may, in certain embodiments, be configured to mount to the cart 103. Alternatively, as described above, the support base 123 may be mounted to another fixture within an operating environment, or may be a standalone component. The robotic arm further includes a first arm joint 125 that is rotatable with respect to the support base 123 around a first rotation axis 127.

A second arm joint 130 is fixed to the first arm joint 125 via a first link 132. The second arm joint 130 is rotatable with respect to the first arm joint 125 and the first link 132 around a second rotation axis 135. In certain embodiments, the second rotation axis 135 is perpendicular to the first rotation axis 127.

A second link 138 attaches a third arm joint 140 to the second arm joint 130. The third arm joint 140 can rotate with respect to the second link 138 around a third rotation axis 143. In certain embodiments, the third rotation axis 143 is parallel with the second rotation axis 135.

A third link 145 extends between the third arm joint 140 and a fourth arm joint 147. The fourth arm joint 147 rotates with respect to the third link 145 around a fourth rotation axis 150. In certain embodiments, the fourth rotation axis 150 is parallel with the second rotation axis 135.

A fourth link 152 joins the fourth arm joint 147 to a fifth arm joint 155. The fifth arm joint 147 rotates with respect to the fourth link 152 around a fifth rotation axis 158. In certain embodiments, the fifth rotation axis 158 is perpendicular to the first rotation axis 127.

A fifth link 160 attached to the fifth arm joint 155 carries a sixth arm joint 163, which rotates with respect to the fifth link 160 around a sixth axis of rotation 165. In certain embodiments, the sixth rotation axis 165 is perpendicular to the fifth rotation axis 158. The sixth arm joint 163 carries sixth link 167. In this embodiment, a working element in the form of a tool assembly 170 extends from a working end 172 of the sixth link, in a direction parallel to the arm's sixth axis of rotation 165.

Please note the configuration of the robotic arm 105 in FIG. 2 is only exemplary, and the exact configuration of the robotic arm 105 can vary considerably in any given embodiment. It may generally be preferred, though, that the robotic arm 105 include elements providing movement with at least six degrees of freedom, to facilitate effective minimally invasive glaucoma surgery (MIGS), or other minimally invasive procedures, at a target site within the patient's eye through a small incision on the eye's outer surface. An operational prototype was built using a model UR5e robotic arm supplied by Universal Robots, a robotic equipment manufacturer with its headquarters in Odense, Denmark (https://www.universal-robots.com).

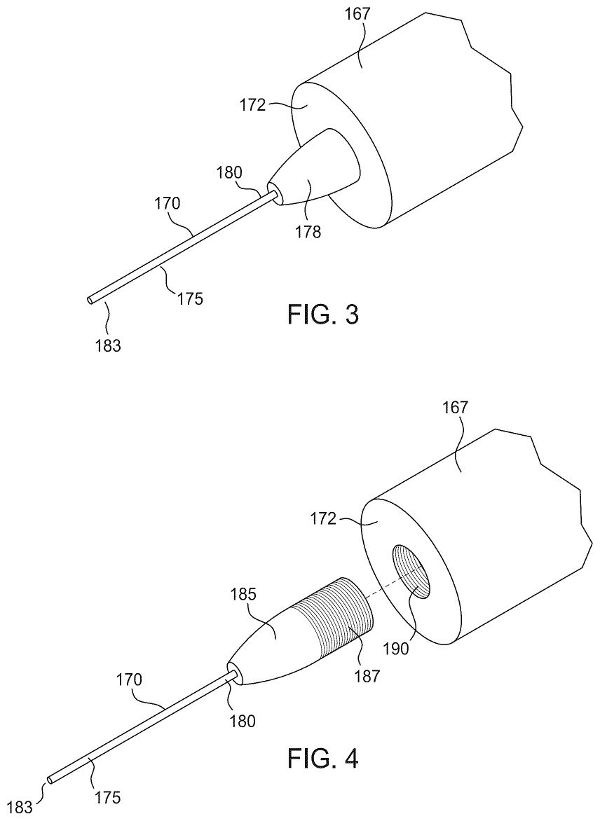

FIG. 3 depicts an exemplary tool assembly 170 which can be coupled at the working end 172 of the sixth robotic arm link 167. The tool assembly includes a narrow elongate support member 175. The elongate support member 175 is fixed to the working end of the sixth arm joint via a mount 178 at a proximal end 180 of the tool assembly. A distal end 183 of the tool assembly's elongate support member 175 can include one or more working elements, as will be described in more detail below.

In certain embodiments, the tool assembly 170 may be fixed permanently at the working end 172 of the sixth arm joint 163. In many cases, though, it may be preferable that the tool assembly 170 be conveniently removable and replaceable at working end 172 of the sixth arm joint 163. This is represented in FIG. 4, which illustrates a tool assembly 170 attachable and removable at the working end 172 via a removable mounting element 185 at the proximal end 180 of the elongate support member 175 of the tool assembly 170. The mounting element 185 in this example is shown as including external threads 187 that interface with corresponding internal threads 190 in the working end 172 of the final robotic arm joint 163, so that the tool assembly 170 can be mounted, held/secured, and removed conveniently with respect to the robotic arm 105.

Please note that the example in FIG. 4 is schematic. Any particular tool assembly 170 and its associated mounting element 185 can be configured appropriately so that the mounting element attaches its tool assembly 170 at the working end 172 of an arm joint with sufficient security, while allowing for removal and replacement of the tool assembly 170 as desired. A threaded assembly is shown, again schematically, in FIG. 4. Other suitable retention assemblies for use with the tool assembly 170 may include sliding and latching mechanisms of various configurations, or other assemblies for retaining the tool assembly 170 on the robotic arm 105, as appropriate in any particular application.

The mechanism for mounting the tool assembly 170 to the robotic arm 105 should locate the distal end 183 of the elongate support member 175 of the tool assembly 170 securely at a precise and repeatable distance away from the working end 172 of the robotic arm 105. An associated control algorithm of the robotic arm 105 computes the position of the distal end 183 based on the rotational positions of the arm's joints, the position and orientation of the arm's links, and/or the known length between the arm's final end (e.g., the working end 172) and the tool assembly's distal end 183. It is thus important that this “known length” be accurately and precision known when using the robotic arm 105. Orientations and positions of the arm's links and arms may be determined via positional encoders integrated within the robotic arm 105.

A tool assembly 170 that is conveniently removable with respect to the robotic arm 105 can allow for the use of different types of tool assemblies 170 for different procedures, for the removal and sterilization of a reusable tool assembly 170 between procedures, for the removal and disposal of a single-use, disposable tool assembly 170 after a completed procedure, and/or for other advantages as appropriate depending on the particular application. It may be preferable in many cases that the removable mounting assembly (e.g., mounting element 185, etc.) allow removal and replacement of different tool assemblies 170 at the working end 172 of the robotic arm 105, by a user of the system 100 manually and without the use of wrenches, screwdrivers, or other additional tools.

Some embodiments may include one or more force and/or torque (F/T) sensors configured to detect force and/or torque at or near the tool assembly 170. Detecting and limiting forces and/or torques at the tool assembly 170 can enhance the patient's safety, by limiting forces applied to the patient's eye by the tool assembly 170 within safe and acceptable limits. Such F/T sensors might be located, for example, in the working end 172 of the robotic arm 105, in the mounting element 185 between the robotic arm 105 and the tool assembly 170, within the elongate member 175 of the tool assembly 170 itself, or elsewhere close enough to the distal end 183 of the tool assembly so as to provide useful information regarding the force/torque applied to the tool assembly 170, and thus, to the eye of the patient.

Some embodiments may include one or more positional encoders or other sensors configured to detect a position and/or orientation of the tool assembly 170 and/or robotic arm 105. Detecting the position and/or orientation of the tool assembly 170 and robotic arm 105 can enhance the patient's safety, by increasing the precision and accuracy of the system during movement and positioning of the robotic arm 105 and tool assembly 170. Such encoders or sensors might be located, for example, in the working end 172 of the robotic arm 105, in the mounting element 185 between the arm 105 and the tool assembly 170, within the elongate member 175 of the tool assembly 170 itself, within the links or joints of the robotic arm 105, or elsewhere in the system so as to provide useful information regarding the position and/or orientation of the robotic arm 150 and tool assembly 170.

Tool assemblies 170 of different effective lengths might be used on a single robotic arm 105. Where that is done, the robotic arm 105 and its associated controller are provided with the effective length of the tool assembly 170, so that the position of the tool assembly 170 and its distal end 183 can be computed and thus “known” by the robotic arm's control mechanisms.

Some embodiments can include tool assemblies 170 that can be driven or otherwise adjusted to lengthen or shorten the tool assembly 170 along an axis parallel to the tool assembly's elongate member 175, while the system is in use. In some cases, this might allow easier or simpler access to a desired location within the eye, in comparison with the simultaneous control and operation of multiple joints of the robotic arm 105 that might otherwise be required.

Some embodiments can include tool assemblies 170 that can be driven or otherwise adjusted to articulate, or bend away from an axis parallel to the tool assembly's elongate member 175, while the system is in use.

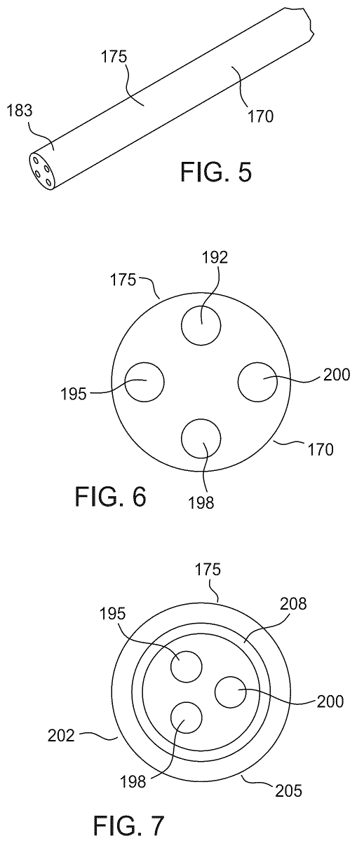

FIG. 5 is an enlarged view of a distal end 183 of a tool assembly 170 like that shown in FIG. 3 or FIG. 4. This particular tool assembly 170 includes ports or locations for four distinct working elements at the tool assembly's distal end 183, as shown in FIG. 6.

FIG. 6 is an end view of the distal end 183 of the tool assembly 170 of FIG. 5. The tool assembly 170 is shown in this configuration as including ports or locations for working elements including an illumination element 192, a visualization element 195, a surgical irrigation and/or aspiration port 198, and an interventional port 200 for delivery of a surgical device or interventional element to a target site inside the eye of a medical patient.

The illumination element 192 delivers visible light or another suitable illumination energy from the distal end 183 of the tool assembly 170 to a surgery site another location of interest inside an eye of the patient 107.

Illumination energy reflected from the target site returns to the visualization element 195, where it is received and passed to other elements of the system for processing and display, e.g., as still images or video for display on display monitors 110.

The illumination energy will in many cases be visible light that is directed onto the desired location, reflected back from the eye's structures and detected either directly, or more commonly after having been passed back along the tool assembly 170 and the robotic arm 105, by a camera, a charge coupled device, or any other suitable apparatus for visible light imaging. The illumination energy might also be infrared light, ultraviolet light, or other useful electromagnetic or other energy. In some cases the illumination element might include apparatus for optical coherence tomography (OCT), or other suitable modes of visualization.

The port 198, in some examples, can be used for the delivery of irrigation fluid to irrigate and clear the area around the target site. In some examples, the port 198 can be used for the delivery of infusion fluid. In such embodiments, the port 198 may be in fluid communication with an irrigation or infusion source/pump disposed in or integrated with the, e.g., the cart 103 or elsewhere in the operating environment. In some examples, excess irrigation fluid, or other fluids, can be aspirated from the eye through this same port (by reversing the pressure to provide suction), or through a separate dedicated suction/aspiration port (which is not depicted separately in this illustration). In such embodiments, the port 198 may be in fluid communication with a vacuum or aspiration pump disposed in or integrated with the, e.g., the cart 103 or elsewhere in the operating environment.

One or more interventional ports 200 can be provided to allow for the delivery of surgical or interventional devices through the same tool assembly. An interventional element might be, for example, a cutter, a laser probe, a delivery element for viscoelastic fluids, a delivery element for placing a stent or a similar implant, or another interventional device of type appropriate for a particular procedure.

Providing these various working elements on and delivering them with a tool assembly 170 mounted in turn at the working end 172 of a robotic arm 105 may be expected to provide various advantages. The delivery and positioning of the working elements may be precisely controlled by carefully controlling the motions of the robotic arm 105. Using the robotic arm 105 can free a hand of the surgeon and relieve fatigue that might otherwise result from the surgeon's needing manually to position and hold the equipment in place. Placing the illumination, visualization, irrigation, and interventional elements at the distal end 183 of a single tool assembly 170 can also put these elements at locations close to one another where they can be used most effectively together.

FIG. 7 is an end view of a possible alternative configuration for working elements at a distal end 202 of a different tool assembly 205. This tool assembly 205 is substantially like that of FIG. 6, in that it includes positions or ports for working elements, including a visualization element 195, an illumination element 192, a surgical irrigation port 198, and an interventional port 200. In the assembly of FIG. 7, though, the illumination element 208 is shown in the form of a ring-light illumination element that surrounds the locations of the other working elements. In some embodiments a larger diameter, ring-shaped illumination element may be found to provide superior illumination for particular applications or for use with certain interventional devices, for example.

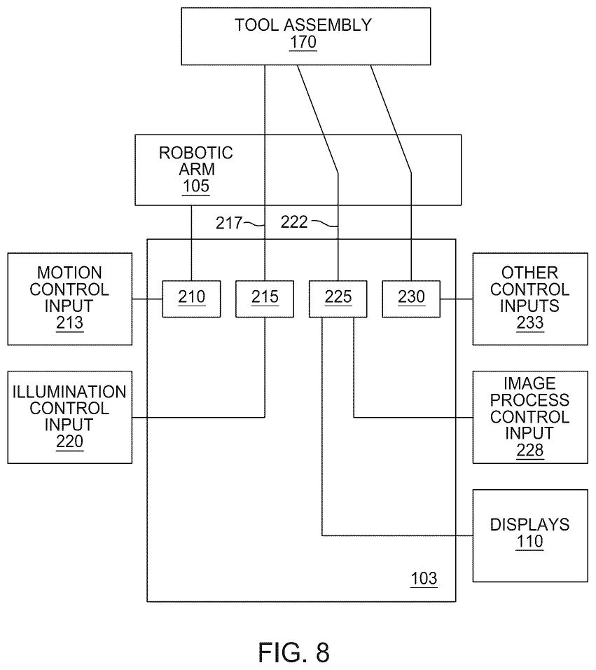

FIG. 8 is a block diagram schematically illustrating various components of the system 100 of FIG. 1. In the example of FIGS. 1 and 8, the cart 103 provides the base and physical support for the robotic arm 105, which in turn carries the tool assembly 170.

The cart 103 further includes a motion controller 210, which is operable to monitor and control movement of the various links of the robotic arm 105 to move the robotic arm 105 and to compute its position and the positions of its various joints and links (e.g., via positional encoders and/or other sensors) of the robotic arm 105, as well as that of the tool assembly 170. The motion controller 210 may receive control inputs from users of the system 100 via a motion control input device 213. During surgical procedures, the motion controller 210 is operable to move the robotic arm 105 so as to extend the distal end 183 and at least a portion of elongate support member 175 of the tool assembly 170 through an incision site in an outer surface of the patient's eye, so that the visualization element 195 and other working elements of the tool assembly 170 are present inside and movable between various positions inside the patient's eye.

The motion control input device 213 may include hand controls such as joysticks, keyboards, or wearable hand-machine interfaces, or foot pedals, voice controls, or other suitable apparatus, which can be pre-programmed to control actuation of the robotic arm 105 via various motions and procedures therewith. Some robotic arms 105 may include sufficient internal feedback devices, such as internal force torque (FT) sensors, so that those robotic arms 105 can be moved as desired by direct movements applied manually to the robotic arms or their corresponding elements by users. Some robotic arms may be provided with force torque sensors operable to respond to the detection of forces or torques applied to elements of the robotic arm above predetermined acceptable limits. Such configurations can help to prevent damage to the patient's eye by preventing the imposition on the eye of unacceptably high forces by the robotic arm.

In some examples, the robotic arm 105 may be autonomously controlled via artificial intelligence (AI) or other automatic control systems.

The cart 103 may also include an illumination source 215. The illumination source 215 can be optically coupled with the illumination element 192, to deliver illumination energy from the illumination source 215 to the illumination element 192 at the distal end 183 of the tool assembly 170.

The illumination source 215 may typically be optically coupled to the illumination element 192 via one or more optical delivery fibers 217. Illumination energy may in some embodiments be delivered to the illumination element 192 via optical delivery fibers disposed inside and along the length of the elongate support member 175 between the proximal and distal ends 180, 183 of the tool assembly 170.

The illumination energy may often be in the form of visible light, but may also include near-infrared or ultraviolet energy, or other illumination energies appropriate to the visualization desired for a given application.

Delivery of illumination energy from the illumination source 215 may be controlled by an illumination control input apparatus 220 operable by the system's users. Those users may use the illumination control input apparatus 220 to adjust, for example, the intensity or type (frequency, etc.) of the illumination energy, as they find most useful in particular circumstances. In certain embodiments, the illumination control input apparatus 220 comprises a physical knob for manually tuning characteristics of the illumination energy, such as a physical knob on a hand controller, foot pedal, cart 103, the surgical console 102, or other suitable device in an operating environment. In certain embodiments, the illumination control input apparatus 220 comprises a digital knob for manually tuning characteristics of the illumination energy, such as a digital control mechanism on a display monitor 110, cart 103, or the surgical console 102.

Illumination energy projected from the illumination source 215 is reflected, from within the patient's eye, back to the visualization element 195 at the distal end 183 of the tool assembly 170. That reflected energy may be transmitted along optical return fibers 222 (or the same optical fiber 217 for delivering the illumination energy) or other appropriate elements to image receiving and processing apparatus 225, which may be housed inside or at the cart 103 or surgical console 102. The image receiving and processing apparatus 225 may include, for example, an active-pixel sensor based on a complementary metal-oxide semiconductor (CMOS) sensor. The optical return fibers 222 may preferably be disposed at least partially inside the elongate support member 175 between the proximal and distal ends 180, 183 of the tool assembly 170, and then further to connect the visualization element 195 to the image receiving and processing apparatus 225.

The visualization device might also be a highly miniaturized camera at the end of the tool assembly, and associated with wires or other means for transmitting signals back for display to the system's users.

Digital signals corresponding to images received at the visualization element 195 are processed and sent to the system's display monitors 110, where they can be seen and used by the surgeon 112, the assistant 115, and others involved in the procedure.

Image process control input apparatus 228 may frequently be present and usable to control processing of the signals before the images are displayed. These controls may be relatively simple. The brightness and contrast of the displayed images might be adjustable by the users, for example.

More complicated adjustment and processing might be used as well, and with various degrees of automation. Displayed images might be stabilized or rotated into orientations more useful to the surgeon. Images from this device might be overlaid or otherwise combined with images from other devices or sensors, including possibly a surgical microscope, an intraoperative OCT system, or other devices used for intraocular procedures.

In certain embodiments, the cart 103 may also carry or house additional apparatus 230 usable with other working elements present at the distal end 183 of the tool assembly 170. This additional apparatus could include, for example pumps, tanks, piping, and supplies for delivering and receiving irrigation fluid for irrigation and/or aspiration at the surgical site or other locations of interest. Additional apparatus might also include apparatus associated with the interventional element(s) deliverable through or operable at that location or port 200 at the distal end 183 of the tool assembly 170. Any of this additional apparatus 230 may be associated with additional control input mechanisms 233 automated or under direct or indirect control of the system's users.

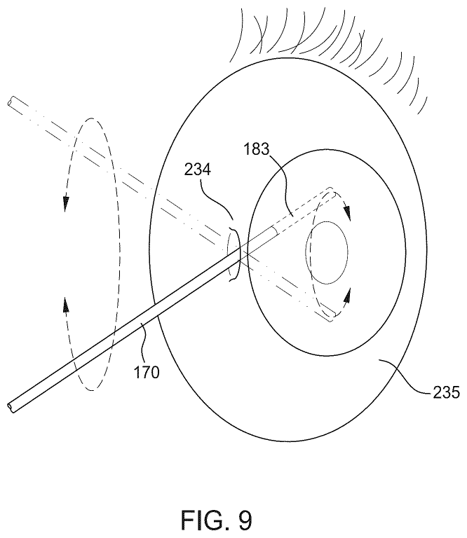

FIG. 9 illustrates a surgery in which the distal end 183 of a surgical tool assembly 170 has been moved into a patient's eye 235 through a small incision site 237. When the robot arm is configured with at least six degrees of freedom, the surgical tool assembly can be moved in and out of the patient's eye and maneuvered around a virtual fixed-point of rotation 239, or a remote center of motion (RCM), at the incision site 237. The working elements at the tool assembly's distal end 183 may thus be maneuvered as required within a substantial working volume in the eye's interior, all while operating through a relatively small incision site 237 just large enough to pass the surgical tool assembly 170 through the incision.

Having a visualization element present inside the eye itself is expected to be safer and to provide superior results, as opposed to other methods in which structures inside the eye including sites for the placement of interventional devices have been visualized more remotely, by instruments outside the eye itself.

Surgical techniques like this, in which instruments are passed into an interior space of a patient's body through a small incision, are often referred to as “keyhole” surgeries. Once the instrument is present in the interior space, the user of the system may then use the robotic arm to maneuver the distal end of the instrument as required within the interior space, without unnecessarily enlarging or otherwise traumatizing the incision site.

The description above has shown, described, and pointed out various features and configurations as applied in various examples. It should be understood, though, that various omissions, substitutions, and changes in the form and details of the example devices can be made without departing from the spirit of the disclosure. It should be understood as well that various features of the type described here can be utilized in various combinations, with individual features included omitted as desired and appropriate. None of these feature should be regarded as required in any particular combination, unless the description clearly requires otherwise. As will be recognized, the elements and combinations described here can be embodied in various forms, some of which may not provide all of the features and benefits described in this disclosure, as some features can be used or practiced separately from others. The scope of protection is therefore defined primarily by the appended claims rather than the foregoing description, and the scope of those claims can be read to include the full scope of equivalents to which those claims are rightfully and legally entitled.

1. A system for intraocular surgery, the system comprising:
a robotic arm;
a tool assembly at a working end of the robotic arm, the tool assembly comprising:
a proximal end at the working end of the robotic arm;
a distal end;
a support member extending between the proximal end and the distal end of the tool assembly; and
a visualization element at the distal end of the tool assembly; and
a motion controller operable to move the robotic arm to extend the distal end and at least a portion of the support member into an eye of a medical patient through an incision site in a surface of the patient's eye, so that the visualization element is located at a position inside the patient's eye.

2. The system of claim 1, wherein the robotic arm is movable with at least six degrees of freedom, and wherein the motion controller is operable to move the robotic arm so that motion of a portion of the support member is constrained at a virtual fixed point of rotation at the incision site in the patient's eye surface.

3. The system of claim 1, further comprising optical return fibers between the visualization element and image receiving and processing apparatus remote from the visualization element.

4. The system of claim 3, wherein the optical return fibers are disposed at least partially inside the support member between the proximal and distal ends of the tool assembly to extend between the visualization element and the image receiving and processing apparatus.

5. The system of claim 1, further comprising image receiving and processing apparatus configured to receive energy from the visualization element and to process that energy to produce digital signals corresponding to that energy for display to a user of the system on a video display monitor.

6. The system of claim 5, further comprising an image process control input apparatus operable by the user to adjust processing of the received energy and production of the digital signals prior to the display on the video display monitor.

7. The system of claim 1, and further comprising an illumination element at the distal end of the tool assembly, wherein the illumination element is configured to delivery illumination energy from the distal end of the tool assembly to a location of interest inside an eye of the patient, and wherein the system is further configured so that illumination energy reflected from the location of interest is received by the visualization element.

8. The system of claim 7, further comprising optical delivery fibers between the illumination element and an illumination source remote from the illumination element.

9. The system of claim 8, wherein the optical delivery fibers are disposed at least partially inside the support member between the proximal and the distal ends of the tool assembly to extend between the illumination element and the illumination source.

10. The system of claim 1, wherein the motion controller is configured to receive input from a user of the system via a motion controller input device that includes at least one of a hand control, a foot control, or apparatus pre-programmed to effect a desired motion of the robotic arm.

11. The system of claim 1, wherein the tool assembly is mounted at the working end of the robotic arm via a removable mounting element configured for mounting, removal, and replacement of the tool assembly on the robotic arm's working end.

12. The system of claim 11, wherein the removable mounting element is configured to allow mounting, removal, and replacement of the tool assembly by a user of the system manually and without an additional tool.

13. The system of claim 1, further comprising an interventional port at the distal end of the tool assembly and configured for delivery of an interventional element to a target site inside the eye of the patient.

14. The system of claim 13, wherein the interventional element comprises at least one of a cutter, a laser probe, a delivery element for viscoelastic fluids, or a delivery element for placing a stent or implant.

15. The system of claim 1, further comprising an irrigation or infusion port at the distal end of the tool assembly and configured for delivery of an irrigation or infusion fluid to a target site inside the eye of the patient.

16. The system of claim 1, further comprising an aspiration port at the distal end of the tool assembly and configured for aspiration of a target site inside the eye of the patient.

17. A system for intraocular surgery, the system comprising:
an adjustable arm;
a tool assembly coupled to a working end of the adjustable arm, the tool assembly comprising a visualization element at a distal end of the tool assembly; and
a motion controller operable to move the adjustable arm to extend the distal end into an eye of a medical patient through an incision site in a surface of the patient's eye, so that the visualization element is located at a position inside the patient's eye, wherein:
the adjustable arm is movable with at least six degrees of freedom, and wherein the motion controller is operable to move the adjustable arm so that motion of a portion of the support member is constrained at a virtual fixed point of rotation at the incision site in the patient's eye surface, and
the motion controller is configured to receive input from a user of the system via a motion controller input device that includes at least one of a hand control, a foot control, or apparatus pre-programmed to effect a desired motion of the robotic arm.

18. The system of claim 17, further comprising optical return fibers between the visualization element and an image receiving and processing apparatus remote from the visualization element, wherein the image receiving and processing apparatus is configured to receive energy from the visualization element and process that energy to produce digital signals corresponding to that energy for display to a user of the system on a video display monitor.

19. The system of claim 18, further comprising an image process control input apparatus operable by the user to adjust processing of the received energy and production of the digital signals prior to the display on the video display monitor.

20. The system of claim 17, further comprising an illumination element at the distal end of the tool assembly, wherein the illumination element is configured to delivery illumination energy from the distal end of the tool assembly to a location of interest inside an eye of the patient, and wherein the system is further configured so that illumination energy reflected from the location of interest is received by the visualization element.
